(19) United States
(12) Patent Application Publication
(10) Pub. No.: US 2010/0274143 A1
(43) Pub. Date: Oct. 28, 2010

(54) METHOD AND APPARATUS FOR ESTIMATING BLOOD PRESSURE

(75) Inventors: Jong Pal KIM, Seoul (KR); Seok Chan KIM, Seoul (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/640,962

(22) Filed: Dec. 17, 2009

(30) Foreign Application Priority Data
Apr. 23, 2009 (KR) 10-2009-0035527

(51) Int. Cl. A61B 5/0225

(52) U.S. Cl. 600/493

(57) ABSTRACT

A method for estimating blood pressure includes: sensing a value of a first sphygmus wave in a region of a user's body while pressurizing the region with a first pressure; sensing a value of a second sphygmus wave in the region while pressurizing the region a second pressure; and estimating blood pressure of the region based on the sensed values of the first sphygmus wave and the second sphygmus wave. The first pressure and the second pressure are each either a variable pressure or a constant pressure. A height of the region, relative to the user's body, is different for the sensing the value of the first sphygmus wave than for the sensing the value of the second sphygmus wave.

### CROSS-REFERENCE TO RELATED APPLICATION

This application claims priority to Korean Patent Application No. 10-2009-0035527, filed on Apr. 23, 2009, and all the benefits accruing therefrom under 35 U.S.C. §119, the content of which in its entirety is herein incorporated by reference.

### BACKGROUND

1) Field

The general inventive concept relates to an apparatus for estimating blood pressure and a method of using the same.

2). Description of the Related Art

Blood pressure is often used as an index of a person's health condition. As a result, various devices for measuring blood pressure are commonly used in medical institutions and in homes. The U.S. Food and Drug Administration (“FDA”) regulates standards applicable to these devices for measuring blood pressure, to ensure compliance with requirements set by the Association for the Advancement of Medical Instrumentation (“AAMI”). More particularly, the American National Standards Institute (“ANSI”)/AAMI SP10, issued by the AAMI, provides specification details, and safety and performance requirements for the devices.

To measure blood pressure, a blood pressure measuring device typically applies pressure to a region through which arterial blood normally flows to stop the flow of the blood in the region, and then slowly reduces the pressure to allow the blood to resume flow. The resulting blood pressure measurement is a systolic blood pressure, which is an instant pressure of an initial sphygmus (e.g., pulse) detected as the pressure is reduced, and a diastolic blood pressure, which is an instant pressure of a final sphygmus.

Other types of blood pressure monitoring devices, such as digital hemadynamometers, for example, calculate blood pressure by detecting a waveform corresponding to a pressure measured while pressurizing a blood vessel.

### SUMMARY

Provided are a method and apparatus for estimating blood pressure, without the requirement of using a characteristic ratio statistically obtained via experimentation. In addition, provided is a computer program product, e.g., a computer readable recording medium, which stores and implements instructions that control a computer to perform the method for estimating blood pressure.

Provided is a method of estimating blood pressure includes: sensing a value of a first sphygmus wave in a region of a user's body while pressurizing the region with a first pressure; sensing a value of a second sphygmus wave in the region while pressurizing the region with a second pressure; and estimating blood pressure of the region based on sensed values of the first sphygmus wave and the second sphygmus wave. The first pressure and the second pressure are each either a variable pressure or a constant pressure, and a height of the region, relative to the user's body, is different for the sensing the value of the first sphygmus wave than for the sensing the value of the second sphygmus wave.

Provided is a computer program product includes a computer readable computer program code which stores and implements a method of estimating blood pressure, and instructions for causing a computer to implement the method. The method includes: sensing a value of a first sphygmus wave in a region of a user's body while pressurizing the region with a first pressure; sensing a value of a second sphygmus wave in the region while pressurizing the region with a second pressure; and estimating blood pressure of the region based on sensed values of the first sphygmus wave and the second sphygmus wave. The first pressure and the second pressure are each either a variable pressure or a constant pressure, and a height of the region, relative to the user's body, is different for the sensing the value of the first sphygmus wave than for the sensing the value of the second sphygmus wave.

Provided is an apparatus for estimating blood pressure includes: a sensing unit which senses a value of a first sphygmus wave in a region of a user's body while pressurizing the region with a first pressure, and which senses a value of a second sphygmus wave in the region while pressurizing the region with a second pressure; an estimator which estimates blood pressure of the region based on sensed values of the first sphygmus wave and the second sphygmus wave; and a user interface which outputs the blood pressure of the region. The first pressure and the second pressure are each either a variable pressure or a constant pressure, and a height of the region, relative to the user's body, is different for the sensing the value of the first sphygmus wave than for the sensing the value of the second sphygmus wave.

### BRIEF DESCRIPTION OF THE DRAWINGS

The above and/or other aspects and features will become apparent and more readily appreciated from the following description, provided with reference to the accompanying drawings, in which:

FIG. 1 is a block diagram of an example of an apparatus for estimating blood pressure;

FIG. 2 is a graph illustrating average thicknesses of portions of a wrist proximate to a radial artery in the wrist;

FIG. 3 is a diagram for describing an example of a method for estimating blood pressure in two positions having different heights, using the apparatus for estimating blood pressure 1 shown in FIG. 1, wherein the apparatus 1 is put on around a user's wrist;

FIG. 4 includes graphs of velocity and pressure versus time illustrating sphygmus waves detected by an example of an apparatus for estimating blood pressure.

FIG. 5 is a graph of voltage versus time illustrating a waveform of a change transmitted from a sensing unit to an example of a voltage determiner;

FIG. 6 is a graph of estimated blood pressures versus time, based on blood pressure calculated by an example of a blood pressure calculator;

FIG. 7 is a diagram for describing an example of using a string connected to a weight for a user to determine a height difference;

FIG. 8 is a diagram for describing an alternative example of obtaining of a height difference by using an arm length;

FIG. 9 is a diagram for describing obtaining of a height difference by using an arm length and an accelerometer sensor according to yet another example;

FIG. 10 is a diagram for describing obtaining of a height difference by using an arm support, according to still another example;

FIG. 11 is a flowchart illustrating an example of a method of estimating blood pressure; and

FIG. 12 is a flowchart illustrating an order of estimating blood pressure of a user by using an example of a method of estimating blood pressure.

### DETAILED DESCRIPTION

The invention now will be described more fully hereinafter with reference to the accompanying drawings, in which various examples are shown. This invention may, however, be embodied in many different forms, and should not be construed as limited to the example set forth herein. Rather, these examples are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular examples only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Examples are described herein with reference to cross section illustrations that are schematic illustrations of idealized examples. As such, variations from the shapes of the illustrations as a result, for example, of manufacturing techniques and/or tolerances, are to be expected. Thus, examples described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

Hereinafter, examples of the present invention will be described in further detail with reference to the accompanying drawings.

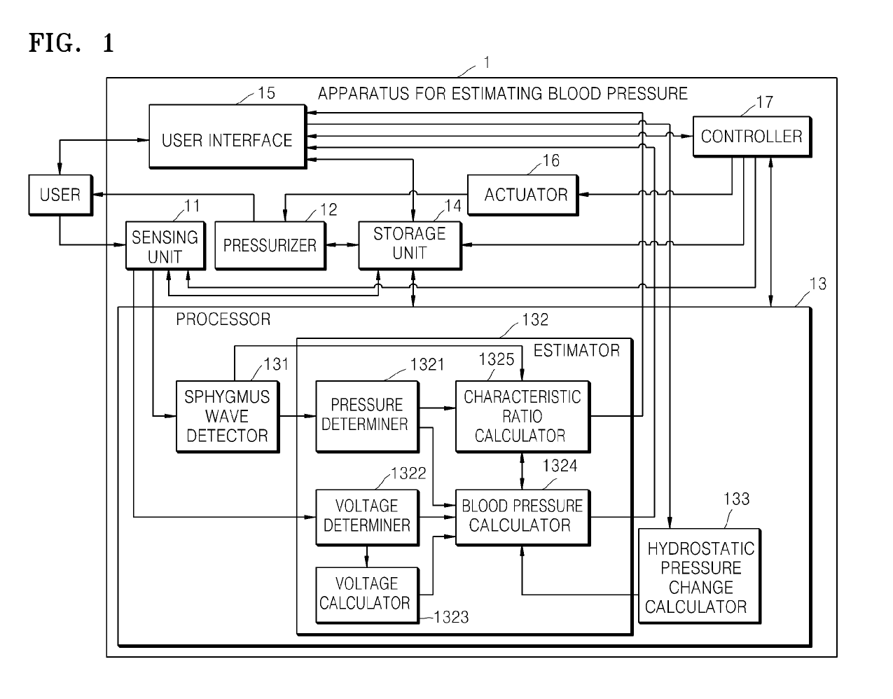

FIG. 1 is a block diagram of an example of an apparatus 1 for estimating blood pressure. Referring to FIG. 1, the apparatus 1 according to an example includes a sensing unit 11, a pressurizer 12, a processor 13, a storage unit 14, a user interface 15, an actuator 16 and a controller 17. The processor 13 includes a sphygmus wave detector 131, an estimator 132 and a hydrostatic pressure change calculator 133. The processor 13 may include, for example, an array of logic gates, or a combination of a general-use microprocessor and a memory in which a program to be executed in the general-use microprocessor is stored, but alternative examples are not limited thereto. For example, the processor 13 may be realized in various forms of hardware including other general-use hardware components neither described herein or illustrated in FIG. 1.

Referring to FIG. 1, the apparatus 1 according to an example includes all instruments and apparatuses for estimating blood pressure, such as a blood pressure instrument, a blood pressure meter, a hemadynamometer and/or a sphygmomanometer, for example.

As used herein, the term blood pressure refers to pressure on walls of blood vessels as blood is pumped out of a heart and flows through the blood vessels. In addition, blood pressure includes arterial blood pressure, capillary blood pressure and venous blood pressure, according to a type of blood vessel in which the blood pressure is measured and/or where the blood vessel in which the blood pressure is measured is located. In addition, the arterial blood pressure, for example, varies according to a user's heartbeat. Additionally, blood pressure further includes systolic blood pressure, e.g., blood pressure corresponding to when blood flows into arteries as ventricles of the heart contract, and diastolic blood pressure, e.g., blood pressure corresponding to affects of the arterial wall due to the elasticity of the arterial wall when the ventricles expand and blood stays in the ventricles.

A sphygmus wave is a wave generated as a sphygmus is transmitted to a peripheral capillary. More specifically, a sphygmus indicates that an artery repetitively expands and relaxes, e.g., contracts, due to the flow of blood through the artery when the heart beats. In other words, when the heart contracts, the blood is supplied to the entire body from the heart via a main artery, and thus pressure in the main artery changes. Such a change of the pressure in the main artery is transmitted to peripheral arterioles of the hands and feet, for example, and a sphygmus wave reflects changes in pressure of the waveform.

In general, blood pressure is estimated using a direct or indirect method, an invasive or noninvasive method, and an intrusive or nonintrusive method, for example. More specifically, the indirect method typically estimates pressure when blood in a brachial artery or a radial artery stops, e.g., is cut off, by winding a blood-pressure cuff around a region at which the blood pressure is to be measured, and applying pressure to the region by injecting air into the blood-pressure cuff. The noninvasive method estimates blood pressure measured outside the blood vessels. Additionally, the intrusive method uses a blood-pressure cuff to estimate blood pressure, while the nonintrusive method estimates blood pressure without using a blood-pressure cuff.

Examples of the noninvasive method include an auscultatory method, an oscillometric method, a tonometric method and a method using a pulse transit time (“PTT”), for example.

The oscillometric method and the tonometric method are typically utilized with a digitalized apparatus for estimating blood pressure. The oscillometric method estimates the systolic pressure and the diastolic pressure by detecting a pulse wave generated in a depressurization process that depressurizes a body part at a constant speed. The detection of the pulse wave is conducted after sufficiently pressurizing the body part through which arterial blood flows to block arterial blood flow. This is similar to a Korotkoff sounds method. The oscillometric method may also be conducted using a pressurization process that pressurizes the body part at a constant speed. A pressure at which the amplitude of a pulse waveform is at a systolic or a diastolic level is thereby estimated as a function of the systolic pressure or the diastolic pressure, as compared with a pressure at which the amplitude of the pulse waveform is at a maximum. The systolic or the diastolic level indicates a systolic or a diastolic characteristic ratio. Alternatively, a pressure at which the amplitude of the pulse waveform varies greatly, relative to variations at other pressures, may be estimated as a function of the systolic pressure or the diastolic pressure. During the depressurization process of the body part at the constant speed after the pressurization process, the systolic pressure is estimated before a point at which the amplitude of the pulse waveform is at the maximum, and the diastolic pressure is estimated after the point at which the amplitude of the pulse waveform is at the maximum. In contrast, in the pressurization process of the body part at the constant speed, the systolic pressure is estimated after the point at which the amplitude of the pulse waveform is at the maximum, and the diastolic pressure is estimated before the point at which the amplitude of the pulse waveform is at the maximum.

To calculate the systolic or the diastolic level of applied pressure, a statistical characteristic ratio may be used. The statistical characteristic ratio is obtained by statistically analyzing sphygmus waves obtained by pressurizing bodies of people during development of a sphygmomanometer. In other words, the pulse amplitude of the sphygmus wave is scaled for the maximum pulse amplitude to be 1, and the mean value of the pulse relative amplitude to the maximum pulse amplitude at the systolic and diastolic blood pressure of the people is calculated as the systolic and diastolic characteristic ratio, respectively. Thus, after manufacturing the sphygmomanometer, when a user operates the sphygmomanometer to measure their blood pressure, the statistical systolic/diastolic characteristic ratio is used to estimate systolic blood pressure and diastolic blood pressure internally in the sphygmomanometer. However, the statistical characteristic ratio may have an error, and thus the blood pressure may not be accurately estimated.

In the tonometric method, blood pressure is measured continuously based on a magnitude and shape of a sphygmus wave generated when a predetermined pressure at which the blood flow in the artery is not completely blocked is applied to the body part.

Types of an apparatuses for estimating blood pressure include a wrist-type hemadynamometer and a finger-type hemadynamometer, depending on which region of the body is to be pressurized. In an example, for example, the apparatus 1 is a wrist-type hemadynamometer, using a user's wrist as a region at which blood pressure will be measured, e.g., estimated, but alternative exampled of the apparatus 1 may be other types of hemadynamometers, such as a finger-type hemadynamometer, for example.

Still referring to FIG. 1, the sensing unit 11 senses a value of a sphygmus wave in the wrist while applying pressures to the wrist at positions having different heights. In an example, the pressures include a first pressure and a second pressure. More specifically, the first and second pressures may be a variable pressure that increases or decreases with a uniform slope, or a constant pressure. In an example, the sensed value of the sphygmus is a value of pressure that changes due to pulses in an internal artery of the wrist. In an example, the variable pressure may be a pressure that continuously changes, either increasing or decreasing, or a series of two or more discrete, short timed constant pressures varying in a stepwise form.

In an example, the sensing unit 11 converts the sensed value into an electric signal, and transmits the electric signal to the sphygmus wave detector 131 and a voltage determiner 1322 of the estimator 132. The electric signal may be a current or a voltage. For purposes of discussion herein, the value of the sphygmus wave will be described as being converted to a voltage. The sphygmus wave includes a dynamic pressure component and a static pressure component. The sensing unit 11 senses the value of the sphygmus wave in the wrist by using at least one sensor. In an example, the sensor may be a pressure sensor, such as a piezoresistive pressure sensor, or a capacitive pressure sensor, but alternative examples are not limited thereto. Rather, the sensor may be any apparatus, device or component which senses a value of a sphygmus wave, in which the value corresponds to a change of pressure in the wrist, and which converts the value into an electric signal such as the voltage or the current, for example.

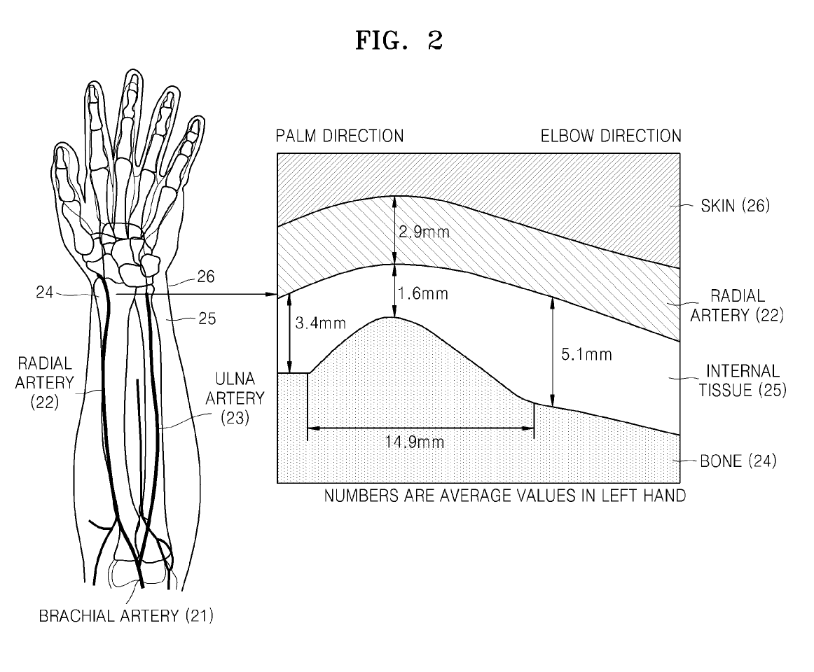

In the wrist-type hemadynamometer, a location for estimating blood pressure may be proximate to a radial artery on a skin surface. FIG. 2 is a graph illustrating average thicknesses, in millimeters (mm) of portions of a user's wrist proximate to a radial artery 22 located in the wrist. Referring to FIG. 2, a brachial artery 21 branches into the radial artery 22 and an ulna artery 23. The apparatus 1 according to an example estimates the blood pressure of the radial artery 22 nearest, e.g., proximate to, a surface of skin 26. Accordingly, while estimating the blood pressure in the blood vessels, e.g., in the radial artery 22, the blood pressure may be affected less than in other regions, such as in internal tissue 25, for example. Referring to the cross section of the wrist shown in FIG. 2, the wrist includes bone 24, the internal tissue 25, and the radial artery 22. A thickness of the internal tissue 25 below the radial artery 22 is the thinnest, compared to other regions, and thus the wrist-type hemadynamometer typically estimates the blood pressure at a location where the radial artery 22 is nearest to the skin surface 26, as shown in FIG. 2.

A changing of a sensed value of the sphygmus wave into a voltage will now be described in further detail. Still referring to FIGS. 1 and 2, in the radial artery 22, the blood pressure transmits pressure around the radial artery 22 as a pressure source. A change of the transmitted pressure corresponds to a value of a first sphygmus wave sensed by the sensing unit 11. The pressure in a local surface above the radial artery 22 is in a linear relationship with the blood pressure in the radial artery 22 since, generally speaking, the actual blood pressure is reduced at a local surface of the skin 26 (as compared to in the radial artery 22, for example). Accordingly, when the pressure at the surface of the skin 26 is determined, the actual blood pressure is estimated by using the linear relationship, which may be represented by Equation 1 below. Thus, in an example, since the change of the value of the sphygmus wave sensed by the sensing unit 11 denotes a change of the pressure on the local surface due to the actual blood pressure in the radial artery 22, the blood pressure in the wrist may be estimated based on the sensed value of the sphygmus wave.

P_{S}=m·BP+n (Equation 1)

In equation 1, P_{S} denotes pressure at a local skin surface, and corresponds to a value of a sphygmus wave sensed by the sensing unit 11, BP denotes the actual blood pressure in the radial artery 22, and m and n are coefficients satisfying a linear relationship between P_{S} and BP. Since m and n change according to conditions associated with pressurizing the radial artery 22 in the wrist, the blood pressure is estimated only when m and n are determined, e.g., are known.

The estimated blood pressure BP has a substantially linear relationship with the pressure P_{S}, and the pressure P_{S} has a substantially linear relationship with the voltage obtained by converting the value of the sphygmus wave sensed by the sensing unit 11 into an electric signal. The linear relationship between the pressure Pₛ and the voltage may be represented by Equation 2 below.

V=a·P_{S}+b (Equation 2)

In Equation 2, V denotes a voltage transmitted from the sensing unit 11, and P_{S} denotes pressure in the local surface, as described above. a denotes sensitivity of a pressure sensor, and b denotes a zero input bias of the pressure sensor. In an example, a and b are constants corresponding to the pressure sensor to transmit a voltage based on, e.g., corresponding to, a pressure, while a and b are predetermined during a calibration process of the pressure sensor, for example.

A relationship between the estimated blood pressure BP and the voltage V transmitted from the sensing unit 11 is represented by Equation 3 below, which is determined by substituting Equation 1 into 2.

V=a·m·BP+a·n+b (Equation 3)

Thus, equation 3 defines the relationship between the voltage V and the estimated blood pressure BP, and may be rearranged as in Equation 4 below.

BP=a·V+β (Equation 4)

In Equation 4, the coefficients of Equation 3 are rearranged to represent the relationship between the voltage V and the estimated blood pressure BP. In Equation 4, α and β are coefficients that defined based on the coefficients used in Equations 1 through 3. In the coefficients used in Equations 1 through 3, a and b are predetermined values, but m and n change according to pressure applied to the wrist, and thus α and β also change according to the pressure applied to the wrist. Referring to Equation 4, the estimated blood pressure BP is determined when α, β, and the voltage V are determined.

As shown in Equations 1 through 4, the sensing unit 11 converts the change of the sensed value of the sphygmus wave into the change of the voltage. The sensing unit 11 transmits the changed voltage to the sphygmus wave detector 131 and the voltage determiner 1322. Thus, in an example, the sphygmus wave has a waveform based on a change of detected blood pressure that is thereafter converted to a voltage signal. The sphygmus wave detector 131 detects the sphygmus wave as a waveform of a voltage change over time. In an alternative example, however, the sphygmus wave may have a waveform of a voltage change according to pressure applied by the pressurizer 12, or a waveform of a change of another voltage signal according to time or pressure. However, for purposes of description herein, the sphygmus wave has the waveform of a voltage change over time.

The sensing unit 11 senses values of sphygmus waves in positions having different heights. In an example, a number of the height positions is at least two, e.g., at least one example includes a first height position and a second height position, and the positions are determined according to a user's selection and/or characteristics of the apparatus 1. Generally, one of the positions, e.g., the first height position, has a same height as a height of the user's heart, while the second height position is at a different height. When values are sensed in the first and second height positions having different height, a value of the estimated blood pressure is compensated for according to a difference of the height, e.g., of the second height position, from the heart. For purposes of description herein, two positions including the first height position having the same height as the heart, and the second height position, having a different height with respect to the heart, will be described, but alternative examples are not limited thereto, e.g., at least one alternative example may include more than two height positions.

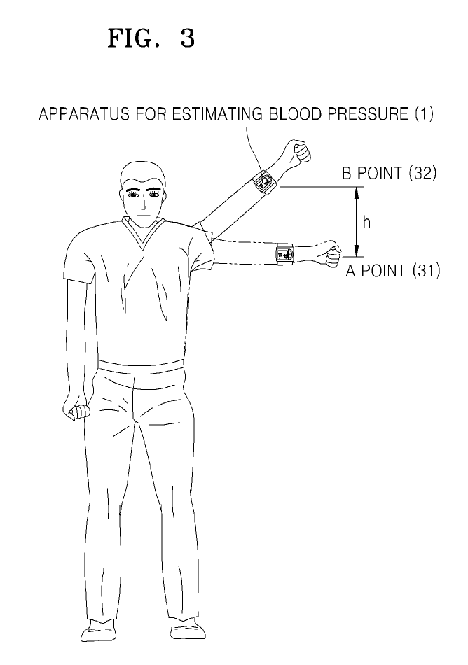

FIG. 3 is a diagram for describing an example of a method for estimating blood pressure in two positions having different heights using the apparatus 1, wherein the apparatus 1 is put on around a user's wrist. The pressure of blood in the bloodstream of the user, which is applied to blood vessels therein, is different due a hydrostatic pressure change, based on a change in height. Hydrostatic pressure indicates pressure acting on a static fluid. Thus, the hydrostatic pressure of blood indicates a pressure of blood pushing against the blood vessel wall in response to the heartbeat. A sphygmus wave dynamically varies, however, since blood in the human body is not a static fluid. However, in an example, the hydrostatic pressure change of blood may be regarded as static pressure change at corresponding points of time when the blood pressure is estimated. The hydrostatic pressure change of blood indicates a difference of pressure according to heights of the positions, e.g., the first height position and the second eight position, and occurs due to the weight of the blood and the height difference between the positions. The hydrostatic pressure change of blood in the artery at the positions having different heights affects the values of the sphygmus waves sensed by the sensing unit 11, and thus estimated blood pressure is also affected by the hydrostatic pressure change. Accordingly, the hydrostatic pressure change of the estimated blood pressure in the positions having different heights is substantially the same as the hydrostatic pressure change of actual blood pressure.

More specifically, a user's bloodstream has potential energy, pressure energy and kinetic energy, for example. In addition, a sum of potential energy, pressure energy and kinetic energy of a fluid having a constant density is constant, according to the law of conservation of energy. Accordingly, based on the law of conservation of energy, the hydrostatic pressure change according to a height difference is identical to a difference between the actual blood pressures at the two positions. Also, as described in greater detail above with reference to Equation 1, the estimated blood pressure BP has a substantially linear relationship with the pressure P_{S} at a local surface of the user's wrist. Accordingly, a difference between the values of the sphygmus waves sensed in each of the positions over a relatively short time is primarily based on the hydrostatic pressure change according to the height difference. Thus, according to an example, a hydrostatic pressure change indicates a hydrostatic pressure change in blood at the two positions having different heights, and is a theoretical value obtained via calculations, as will be described in greater detail below.

Referring to FIG. 3, when the user wears the apparatus 1 and estimates blood pressure at positions A and B, which in an example correspond to a first position 31 and a second position 32, the sensing unit 11 in the apparatus 1 senses values of sphygmus waves at each of the A and B positions, e.g., a first sphygmus wave and a second sphygmus wave sensed at the first position 31 and the second position 32, respectively. The sensing unit 11 in the apparatus 1 according to an example senses values of the sphygmus waves, e.g., of the first sphygmus wave at the A position 31, (the first position 31), which has a substantially same height as the user's heart, by extending the arm straight, and then senses values of sphygmus waves, e.g., of the second sphygmus wave, at the B position 32,(the second position 32), which is at a height different, e.g., higher than the height of the heart, by raising the arm. Thus, in an example, a hydrostatic pressure change between a first pressure and a second pressure is generated, since the heights of the A position 31 (corresponding to the first pressure example) and the B position 32 (corresponding to the second pressure, for example) are different by a value h. Accordingly, the blood pressure is estimated by using the hydrostatic pressure change and the values of the first and second sphygmus waves sensed at each of the A and B positions 31 and 32, respectively. In alternative examples, locations of the A and B positions 31 and 32 may vary, and an order of sensing values of the first and second sphygmus waves at the A and B positions 31 and 32, respectively, may change, e.g., a sphygmus wave at the B position 32 may be sensed before a sphygmus wave at the A position 31 is sensed, for example. An example of method of calculating a hydrostatic pressure change and using a difference between the hydrostatic pressure change and a voltage will be described in further detail below.

Referring again to FIG. 1, the pressurizer 12 pressurizes the user's wrist before the sensing unit 11 senses the values of the sphygmus waves in the wrist. Examples of a method of pressurizing the wrist according to an example include an entire pressurizing method using a blood-pressure cuff, and a partial pressurizing method that pressurizes a part region of the wrist, for example. The actuator 16 adjusts the pressure of the pressurizer 12 applied to the wrist. More particularly, the actuator 16 determines a variable pressure that uniformly increases or decreases, or, alternatively, a constant pressure, to be applied to the wrist. In alternative examples, the apparatus 1 may use other pressurizing methods.

In an example, the sensing unit 11 senses values of sphygmus waves from before or at the time the pressurizer 12 pressurizes the wrist and until the pressurizer 12 stops pressurizing the wrist. The sensing unit 11 then transmits to the sphygmus wave detector 131 a value of a first sphygmus wave sensed while pressurizing the wrist with the variable pressure, e.g., the first pressure, and transmits to the voltage determiner 1322 a value of a second sphygmus wave sensed while pressurizing the wrist with the constant pressure, e.g., the second pressure corresponding to when the pressurizer 12 stops pressurizing the wrist. The actuator 16 determines one of the variable pressure and the constant pressure to be applied to the wrist, as well as a rate of increasing the variable pressure and/or a magnitude of the constant pressure, either or both of which may be set by the user according to a usage environment, for example. In an example, the constant pressure is a pressure applied so as not to occlude blood vessels, and, more specifically, is a pressure lower than a mean arterial pressure (“MAP”), determined based on the sphygmus waves. In an example, the MAP is a pressure applied at a point of time when the sphygmus wave is expected to have a maximum pulse amplitude when the variable pressure is applied to the wrist. Moreover, the pressure applied at a point of time when the sphygmus wave is expected to have the maximum amplitude is substantially the same as the actual blood pressure. Accordingly, the MAP is substantially the same as the actual blood pressure. A time for applying pressure is set to be between a point of time when the artery bloodstream stops and a point of time when the artery bloodstream circulates normally. When the wrist is pressurized at different heights, the rate of increasing the variable pressure and the size of the constant pressure are set to be substantially the same.

In an example, the user may determine how the wrist is to be pressurized, as well as a measuring sequence for pressurizing the wrist, according to inputs provided to a user input interface, for example. In an example, how the wrist is to be pressurized and the order are determined according to equations and a method calculated by a blood pressure calculator 1324. In other words, the pressurizer 12 according to an example may determine whether the variable pressure, the constant pressure, or both the variable pressure and the constant pressure are to be applied to the wrist at each position. Also, when the pressurizer 12 determines to apply both the variable pressure and the constant pressure, the pressurizer 12 may also determine which one of the variable pressure and the constant pressure is to be applied first. For example, the variable pressure may be applied only at one position, and the constant pressure may be applied at both positions. Alternatively, the variable pressure and the constant pressure may be applied at both positions, but alternative examples are not limited thereto.

Referring again to FIG. 3, according to an example, the sensing unit 11 senses the value of the first sphygmus wave while the pressurizer 12 pressurizes the wrist with variable pressure at the A position 31, and then senses the value of the second sphygmus wave while the pressurizer 12 pressurizes the wrist with the constant pressure at the A position 31 and the B position 32. According to an alternative example, the sensing unit 11 senses the value of the first sphygmus wave while the pressurizer 12 pressurizes the wrist with variable pressure at the A position 31, senses the value of the second sphygmus wave while the pressurizer 12 pressurizes the wrist with the constant pressure at the A position 31, and then senses another value of the second sphygmus wave while the pressurizer 12 pressurizes the wrist at the B position 32 under substantially the same conditions as at the A position 31. In other words, in alternative examples, the user determines how the wrist is to be pressurized, as well as the order of positions for pressurizing the wrist.

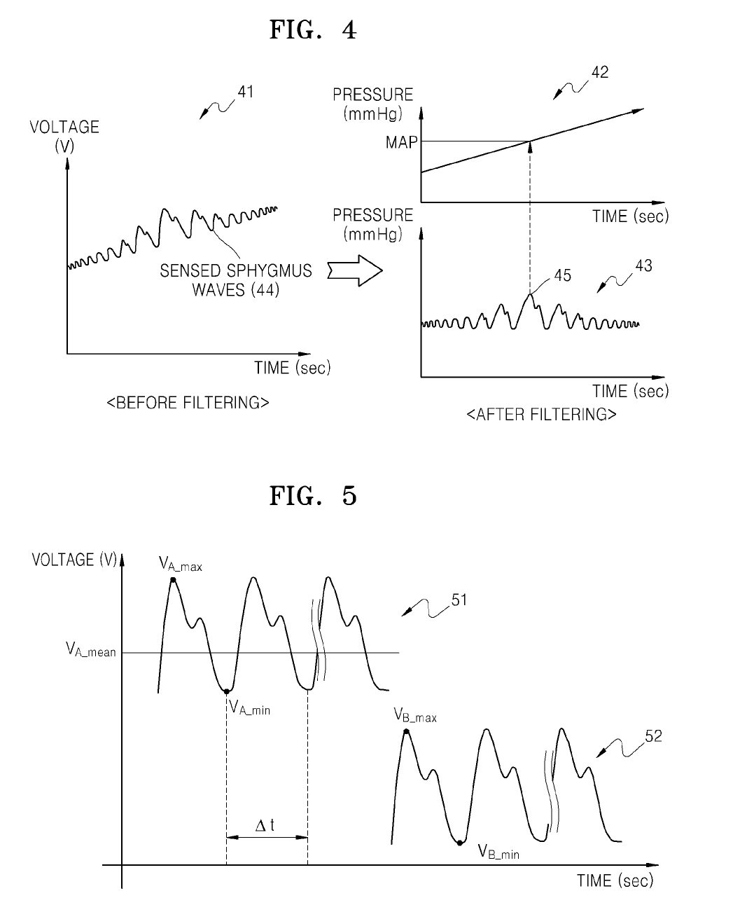

The sphygmus wave detector 131 detects sphygmus waves, such as the first and second sphygmus waves, but not being limited thereto, based on voltages converted in the sensing unit 11. More particularly, the sphygmus waves detected by the sphygmus wave detector 131 include a sphygmus wave that passes through a high pass filter (“HPF”) and a sphygmus wave that passes through a low pass filter (“LPF”), for example. As shown in FIGS. 4 and 5, detected sphygmus waves have a waveform of a pressure change over time. In an example, the sphygmus wave detector 131 uses Equation 2, above, to generate waveforms of the detected sphygmus waves. A form of the detected sphygmus waves is different according to a pressure applied by the pressurizer 12. In other words, the form of the sphygmus waves is different based on whether the variable pressure or the constant pressure is applied. Specifically, when the variable pressure is applied to the wrist, the sphygmus wave detector 131 transmits the detected sphygmus waves to a pressure determiner 1321. When the user selects to calculate the blood pressure by using a characteristic ratio, calculated by a characteristic ratio calculator 1325, the sphygmus wave detector 131 transmits the detected sphygmus waves to the characteristic ratio calculator 1325.

More specifically, the sphygmus wave detector 131 detects sphygmus waves in each band by filtering voltages received from the sensing unit 11 using a HPF and a LPF. For the filtering, any suitable HPF and LPF are used, and a detailed description thereof will be omitted or simplified.

FIG. 4 includes graphs of velocity and pressure versus time illustrating sphygmus waves detected by an example of an apparatus for estimating blood pressure. More particularly, FIG. 4 illustrates sphygmus waves detected by the sphygmus wave detector 131 while the pressurizer 12 pressurizes the user's wrist with the variable pressure according to an example. Referring to FIG. 4, graph 41 shows sphygmus waves, detected at one position, e.g., either the first position 31 or the second position 32 (FIG. 3), before being filtered, while graph 42 shows the sphygmus waves after being filtered by a LPF, and graph 43 shows the sphygmus waves after being filtered by a HPF. The sphygmus waves in graphs 42 and 43 have waveforms of a pressure change over time. A waveform 44 is shown corresponding to when the pressurizer 12 applies the variable pressure (that uniformly increases, for example) to the user's wrist. The waveform 44 is a waveform of a voltage change over time that is transmitted from the sensing unit 11. Also, as described in greater detail above, the sphygmus wave detector 131 converts the pressure into a voltage by using Equation 2, and detects the sphygmus waves shown in graph 42 and the sphygmus wave shown in graph 43. The change of the sphygmus waves of the graph 42, wherein a low frequency band of the sphygmus waves is filtered by the LPF, shows pressure applied to the wrist.

The estimator 132 according to an example includes the pressure determiner 1321, the voltage determiner 1322, a voltage calculator 1323, the blood pressure calculator 1324 and the characteristic ratio calculator 1325.

The pressure determiner 1321 determines the MAP from the sphygmus waves of the graphs 42 and 43 detected by the sphygmus wave detector 131. The pressure determiner 1321 transmits the determined MAP to the blood pressure calculator 1324. When the user selects to calculate the blood pressure by using the characteristic ratio calculated by the characteristic ratio calculator 1325, the pressure determiner 1321 transmits the determined MAP to the characteristic ratio calculator 1325. As discussed above, the MAP is pressure applied to the wrist at a time when the sphygmus waves of the graph 43, which is detected by being filtered by the HPF, are expected to have a maximum amplitude. The pressure determiner 1321 determines the MAP only at one height position or, alternatively, at two or more height positions, according a method of calculating blood pressure in the blood pressure calculator 1324.

Referring again to FIG. 4, the pressure applied at a time point 45 when the sphygmus waves of the graph 43, filtered by the HPF, are expected to have the maximum amplitude is MAP. In an example, the applied pressure is pressure applied at the same point of time as the time point 45 on the sphygmus waves of the graph 42 filtered by the LPF. Alternatively, instead of using the time point 45, a value obtained by interpolating peaks of the filtered sphygmus waves in the graph 43 may be used, wherein the peaks are in a section between peaks just before the maximum peak and/or peaks right after the maximum peak. Also, the MAP may be determined by using pressure applied at a time of one of the interpolated value and the maximum amplitude that has a bigger value. In this case, a time of the interpolated value is used since a peak of sphygmus waves before the maximum peak or a peak of sphygmus waves after the maximum peak may be the maximum in the sphygmus waves of the graph 43 that is filtered with the HPF

The voltage determiner 1322 according to an example determines voltages for one period, e.g., a first period, of the sphygmus waves, from among voltages corresponding to values of all the sphygmus waves sensed by the sensing unit 11, while pressurizing the wrist with the constant pressure. Thus, when voltages of the one period are determined at each height position, starting points of the one period of the points are set to correspond to each other so that the forms of the waveform of the points are substantially the same. Generally, the corresponding starting points may be set to be the maximum voltage or, alternatively, the minimum voltage from among the voltages corresponding to the sensed values, but the corresponding starting points in alternative examples are not limited thereto.

In addition, upon determining the voltages of one period in each of the first height position and the second height position, the voltage determiner 1322 determines voltages corresponding to each other from among a plurality of the voltages of one period determined for each height position. However, the voltage determiner 1322 may not determine the voltages corresponding to each other, according to an alternative example of a method of calculating blood pressure in the blood pressure calculator 1324. In this case, when the times of the periods determined in each height position are the same, as discussed above, the voltages corresponding to each other are voltages after the same time has passed from the starting point of the first period. However, when the times of the periods determined in each point are not the same, the voltage determiner 1322 normalizes the time of one period to a value of 1, and then determines the voltages corresponding to each other at the location when the normalized time is substantially the same.

The voltage determiner 1322 transmits the voltages corresponding to the values of the sphygmus waves, the values received from the sensing unit 11, to the blood pressure calculator 1324, and also transmits the voltages corresponding to each other, the voltages determined in the voltage determiner 1322, to the blood pressure calculator 1324. Additionally, the voltage determiner 1322 transmits the voltages of one period to the voltage calculator 1323.

FIG. 5 is a graph of voltage versus time illustrating a waveform of a change transmitted from the sensing unit 11 to the voltage determiner 1322, while pressurizing the wrist with the constant pressure, according to an example. Referring to FIG. 5, the graph therein shows a waveform of the voltage change transmitted from the sensing unit 11, after pressurizing the wrist with the same pressure at each height position, e.g., at the first height position and the second height position. Waveform 51 in FIG. 5 shows voltages corresponding to values of sphygmus waves sensed at a lower height position than for waveform 52. In other words, the waveforms 51 and 52 have different voltages, since the actual blood pressure at the upper and lower height positions is different by the hydrostatic pressure change of blood discussed above. In waveforms 51 and 52, voltages having the same waveform are repeated by a uniform time Δt. In an example, the uniform time Δt denotes one period of the sphygmus waves. In an example, the voltages having the same waveform are repeated because the wrist is pressurized with the constant pressure. Thus, the voltage change during the uniform time Δt changes according to the actual blood pressure that changes when the heart beats, e.g., contracts and relaxes, once.

Referring still to FIG. 5, the voltage determiner 1322 determines voltages of one period from the waveform 51, and determines voltages of one period from the waveform 52. In an example, starting points of one period are set to locations that correspond to each other, and the starting points may be the maximum voltages or, alternatively, the minimum voltages. The determined voltages of one period are transmitted to the voltage calculator 1323. Additionally, the voltage determiner 1322 determines voltages that correspond to each other from among the voltages of one period in the waveform 51 and the voltages of one period in the waveform 52. For purposes of description herein, the corresponding voltages are the maximum or, alternatively, the minimum voltages, but the corresponding voltages in alternative examples are not limited thereto. The voltage determiner 1322 transmits the voltages of one period to the voltage calculator 1323, and transmits the voltages corresponding to each other to the blood pressure calculator 1324.

In an example, the user may determine whether the voltage determiner 1322 determines the maximum voltage or, alternatively, the minimum voltage as the corresponding voltages, based on a usage environment, for example. Alternatively, as described above, the user may determine whether the voltage determiner 1322 determines other voltages as the corresponding voltages instead of the maximum or minimum voltages.

When the voltage determiner 1322 determines that the voltages of one period are voltages of an initial period, as illustrated in FIG. 5, the voltage determiner 1322 determines the maximum voltage V_{A}_{_{—}}ₘₐₓ in the one period of the waveform 51, and the maximum voltage V_{B}_{_{—}}ₘₐₓ in the one period of the waveform 52. Alternatively, the voltage determiner 1322 may determine the minimum voltage V_{A}_{_{—}}ₘᵢₙ in the one period of the waveform 51, and the minimum voltage V_{B}_{_{—}}ₘᵢₙ in the one period of the waveform 52.

The voltage calculator 1323 calculates a mean voltage Vₘₑₐₙ (e.g., an A position (31) mean voltage V_{A}_{_{—}}ₘₑₐₙ, as shown in FIG. 5, of the voltages of one period determined by the voltage determiner 1322. The voltage calculator 1323 calculates the mean voltage Vₘₑₐₙ of the voltages of one period for at least one of the two height positions. In an example, the mean voltage Vₘₑₐₙ may be calculated by using Equation 5 below. Then, the voltage calculator 1323 transmits the calculated mean voltage Vₘₑₐₙ to the blood pressure calculator 1324.

$${V}_{mean} = \frac{1}{Δ t} {\int }_{Δ t} V d t \qquad ( Equation 5 )$$

In Equation 5, the mean voltage Vₘₑₐₙ may be calculated by dividing a value ∫_{Δt}^{Vdt} obtained by integrating the voltage change for a time Δt of one period by the time Δt.

When the pressure determiner 1321 determines one MAP, e.g., the MAP for the A position 31, the voltage calculator 1323 calculates one mean voltage Vₘₑₐₙ, e.g., the A position (31) mean voltage V_{A}_{_{—}}ₘₑₐₙ. However, when the pressure determiner 1321 determines the MAPs at each height position, the voltage calculator 1323 calculates the mean voltages Vₘₑₐₙ at each height position. Thus, the number of determined MAPs and calculated mean voltages Vₘₑₐₙ are determined based on the method of calculating the blood pressure of the blood pressure calculator 1324, e.g., the number of different height positions. However, in an alternative example, the mean voltage Vₘₑₐₙ is calculated only at one height position, the voltage calculator 1323 may calculate the mean voltage Vₘₑₐₙ at a height position other than the position where the MAP is determined.

As shown in FIG. 5, the voltage calculator 1323 calculates, at the A height position 31 (FIG. 3) the mean voltage Vₘₑₐₙ of the voltages in the time Δt by using Equation 5. Also, although not illustrated in FIG. 5, the voltage calculator 1323 may calculate the mean voltage Vₘₑₐₙ at the B height position 32 of FIG. 3, e.g., a B height position (32) mean voltage V_{B}_{_{—}}ₘₑₐₙ (not shown). When one MAP is determined, the voltage calculator 1323 calculates a mean voltage V_{A}_{_{—}}ₘₑₐₙ at the A height position 31 or a mean voltage V_{B}_{_{—}}ₘₑₐₙ (not shown) at the B height position 32. However, when the MAPs are determined at both A and B height positions 31 and 32, respectively, the voltage calculator 1323 calculates both the mean voltages V_{A}_{_{—}}ₘₑₐₙ and V_{B}_{_{—}}ₘₑₐₙ.

The blood pressure calculator 1324 calculates the blood pressure by using the pressure determined by the pressure determiner 1321 and the values of first and second sphygmus waves sensed while pressurizing the wrist with the constant pressure. More specifically, the blood pressure calculator 1324 calculates the blood pressure by using the MAP determined by the pressure determiner 1321, the voltages corresponding to the values of the sphygmus waves sensed by the sensing unit 11, the voltages corresponding to each other determined by the voltage determiner 1322, and the mean voltage calculated by the voltage calculator 1323. However, when the voltage determiner 1322 does not determine the voltages corresponding to each other, as discussed above, the voltages corresponding to each other are not be used to calculate the blood pressure. When the hydrostatic pressure change calculator 133 calculates the hydrostatic pressure changes at the height positions having different heights, the calculated hydrostatic pressure changes are transmitted to the blood pressure calculator 1324. The estimator 132 thereby estimates the blood pressure calculated by the blood pressure calculator 1324 as actual blood pressure in the radial artery of the user's wrist.

Hereinafter, an example of a method of calculating blood pressure by using the MAP determined at one height position, the voltages corresponding to each other at different height positions, the mean voltage calculated at one height position and the calculated hydrostatic pressure change will be described in further detail. Thereafter, an alternative example of a method of calculating of blood pressure by using the MAPs and the mean voltages calculated at each height position will be described in further detail.

To calculate estimated blood pressure BP by using Equation 4, α and β are calculated first. In Equation 4, the voltage V corresponds to a value of a sphygmus wave sensed by the sensing unit 11 and is transmitted to the blood pressure calculator 1324 via the voltage determiner 1322, as described in greater detail above. α and β are calculated using equations that will be described below. Also, for purposed of description, an A height position is hereinafter defined as the lower height position, and a B height position is the higher height position, but alternative examples are not limited thereto.

In an example, in the calculating of the blood pressure by using the MAP determined at one height position, the voltages corresponding to each other at the first and second height positions, the mean voltage calculated at one height position, and the calculated hydrostatic pressure change are determined, as will now be described in further detail.

According to an example, α is calculated using the hydrostatic pressure change calculated by the hydrostatic pressure change calculator 133, and a difference between two maximum or, alternatively, two minimum voltages corresponding to each other at two height positions. However, as described above, other voltages corresponding to each other may be used instead of the maximum (or minimum) voltages. A difference between estimated blood pressure of the wrist at the A and B height positions for a short time is substantially the same as the hydrostatic pressure change, as described in greater detail above. Accordingly, the difference may be expressed by Equation 6 below.

BP_{A}−BP_{B}=ρgh (Equation 6)

In Equation 6, BP_{A} denotes estimated blood pressure at the A height position, e.g., the first height position, BP_{B} denotes estimated blood pressure at the B height position, e.g., the second height position, and a difference between BP_{A} and BP_{B} is substantially the same as a hydrostatic pressure change ρgh according to a height difference h between the A and B height positions. In Equation 6, ρ denotes a blood density of a user and g denotes a gravitational acceleration constant. By using Equations 4 and 6, the hydrostatic pressure change may be expressed as a voltage, instead of the estimated blood pressure BP_{A} and BP_{B}, as shown in Equation 7 below.

αV_{A}+β−(αV_{B}+β)=ρgh (Equation 7)

In Equation 7, V_{A} and V_{B} denote voltages corresponding to each other at each of the A and B height positions, respectively. Equation 8, below, is generated by rearranging Equation 7, and thus, α may be calculated by using Equation 8. A specific equation used to calculate a (from among Equation 8) may be set according to a usage environment, for example.

$$α = \frac{ρ gh}{{V}_{A} - {V}_{B}} , \\ α = \frac{ρ gh}{{V}_{A\_max} - {V}_{B\_max}} , \\ α = \frac{ρ gh}{{V}_{A\_min} - {V}_{B\_min}} \qquad ( Equation 8 )$$

More particularly, in the first equation of Equation 8, V_{A} and V_{B} denote voltages corresponding to each other, as determined by the voltage determiner 1322, and the hydrostatic pressure change ρgh is a value calculated by the hydrostatic pressure change calculator 133. V_{A} and V_{B} are voltages corresponding to each other, which include the maximum (or minimum) voltage, determined at height positions A and B, respectively. The second and third equations of Equation 8 are more detailed versions of the first equation of Equation 8. More specifically, the second equation of Equation 8 is used to calculate a by using the maximum voltage, and the third equation of Equation 8 is used to calculate a by using the minimum voltage. In an exemplary, α is calculated by using the maximum (or minimum voltage), but in alternative examples, α may be calculated by using other voltages corresponding to each other the A and B height positions, respectively.

An example of a method of calculating β will now be described in further detail. The MAP determined by the pressure determiner 1321, as discussed above, is substantially the same as the actual blood pressure of the user's wrist. Also, a central voltage of one period of sphygmus waves corresponds to the mean voltage Vₘₑₐₙ. Accordingly, the MAP and the mean voltage Vₘₑₐₙ correspond to each other, and thus the MAP and the mean voltage Vₘₑₐₙ have a substantially linear relationship, which is obtained using Equation 4, above. Since the estimated blood pressure BP has a substantially linear relationship with the voltage V corresponding to the value of the sphygmus wave sensed by the sensing unit 11, and the MAP corresponds to the mean voltage Vₘₑₐₙ, Equation 9, below, may be generated.

MAP=α·Vₘₑₐₙ+β (Equation 9)

IN Equation 9, MAP denotes the MAP determined by the pressure determiner 1321, and Vₘₑₐₙ denotes a mean voltage calculated by the voltage calculator 1323. Additionally, α is calculated by using Equation 8, described in greater detail above. Accordingly, since all other values except β are calculated in Equation 9, Equation 10 may be used to calculate (3, by arranging Equation 9.

β=MAP−α·Vₘₑₐₙ (Equation 10)

Equation 10 is generated by rearranging Equation 9 with respect to β. In Equation 10, the mean voltage Vₘₑₐₙ may not be a mean voltage calculated at the same point as the MAP. In this case, the blood pressure BP is calculated by using α, β, and the voltages corresponding to the values of sphygmus waves sensed by the sensing unit 11 while pressurizing the user's wrist with the constant pressure in Equation 4. The calculated blood pressure is estimated to be the actual blood pressure of the radial artery in the wrist.

An example of a method of calculating blood pressure by using the MAPs and the mean voltages determined at each height position will now be described in further detail. In an alternative example, another method for obtaining α and β is included. More particularly, the alternative example is different from the previously described examples in that the blood pressure calculator 1324 does not obtain the hydrostatic pressure change from the hydrostatic pressure change calculator 133. Accordingly, in the alternative example, a separate method or apparatus for measuring a height difference between two height positions is not required, and the user may not locate the wrist at the two different height positions having the height difference therebetween, as will be described in further detail below.

In an alternative example, the pressure determiner 1321 determines two MAPs at each of the two height positions, e.g., at both the first height position and the second height position. When the two height positions are A and B height positions, respectively, MAP_{A} denotes the MAP at the A height position and MAP_{B} denotes the MAP at the B height position.

Additionally, the voltage determiner 1322 according to an alternative example determines voltages of one period of the sphygmus waves at each of the A and B height positions. Then, the voltages of one period at each of the A and B height positions are transmitted to the voltage calculator 1323, and the voltage calculator 1323 calculates two mean voltages Vₘₑₐₙ at the two height positions, e.g., a mean voltage V_{A}_{_{—}}ₘₑₐₙ at the A height position, and a mean voltage V_{B}_{_{—}}ₘₑₐₙ at the B height position are calculated.

Equation 11, below, is generated by replacing the MAPs and the mean voltages in Equation 9, above.

MAP_{A}α·V_{A}_{_{—}}ₘₑₐₙ+β,

MAP_{B}α·V_{B}_{_{—}}ₘₑₐₙ+β (Equation 11)

Equation 12 is generated by combining the two equations of Equation 11.

$$α = \frac{{MAP}_{A} - {MAP}_{B}}{{V}_{A\_mean} - {V}_{B\_mean}} \qquad ( Equation 12 )$$

In an example, α may be calculated by using Equation 12. When Equation 12 is used, α is calculated without using a hydrostatic pressure change.

Equation 10 may thereafter be used to calculate β. In this case, β may be calculated by using the MAP and the mean voltage at the same height position, based on the equation from which α is calculated, or, alternatively, by using the MAPs and the mean voltages at different height positions. Then, the blood pressure BP is calculated by using α, β and the voltages corresponding to the values of sphygmus waves sensed by the sensing unit 11 while pressurizing the wrist with the constant pressure in Equation 4. The calculated blood pressure is estimated to be the actual blood pressure of the radial artery in the wrist.

According to a usage environment, for example, α and β may be calculated using Equations 8 through 10 or, alternatively, using Equations 10 and 12, according to the examples described herein. It will be noted that, in alternative examples, however, that α and β may be calculated by using different methods and/or combinations of equations described herein.

Thus, in an example, when α and β are calculated, the blood pressure calculator 1324 calculates the blood pressure BP by using Equation 4, and the estimator 132 estimates the calculated blood pressure BP as the actual blood pressure of the user. The user interface 15 obtains the calculated blood pressure, and outputs the calculated blood pressure having the maximum value as a systolic blood pressure, and the calculated blood pressure having the minimum value as a diastolic blood pressure. Also, the user interface 15 may calculate a mean of the change of calculated blood pressure, and output the mean blood pressure.

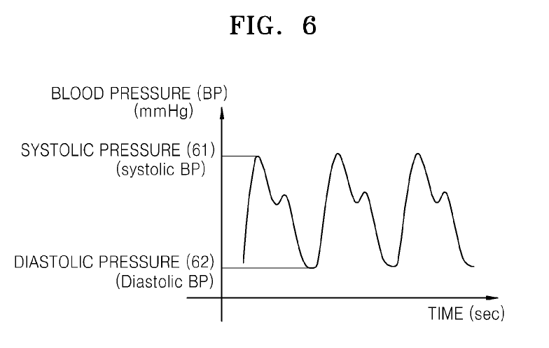

FIG. 6 is a graph of a estimated blood pressures BP versus time t, based on blood pressure calculated by the blood pressure calculator 1324, according to an example. Referring to FIG. 6, blood pressure having the maximum value is referred to as a systolic blood pressure 61, and blood pressure having the minimum value is referred to as a diastolic blood pressure 62.

In an example, the characteristic ratio calculator 1325 calculates a characteristic ratio of blood pressure of the user using the values of sphygmus waves, e.g., the first sphygmus wave, sensed in the wrist while pressurizing the wrist with the variable pressure, and the blood pressure calculated by the blood pressure calculator 1324. Then, when values of sphygmus waves are newly sensed, e.g., the second sphygmus wave, while pressurizing the wrist with the variable pressure, the characteristic ratio calculator 1325 calculates systolic blood pressure and diastolic blood pressure of the wrist based on the sensed values of the first and second sphygmus waves, a newly determined MAP, and the pre-calculated characteristic ratio. The estimator 132 estimates the systolic blood pressure and diastolic blood pressure calculated by the characteristic ratio calculator 1325 as actual systolic blood pressure and actual diastolic blood pressure of the user. In other words, the blood pressure of the user is estimated via an oscillometric method, based on the calculated characteristic ratio. The user interface 15 outputs the systolic blood pressure and the diastolic blood pressure calculated and estimated by the characteristic ratio calculator 1325 and the estimator 132. Since the characteristic ratio calculated by the characteristic ratio calculator 1325 is calculated based on the blood pressure calculated by the blood pressure calculator 1324, the characteristic ratio according to an example is substantially more accurate than a conventional statistical characteristic ratio. The storage unit 14 stores the calculated characteristic ratio.

In an example, the blood pressure having the maximum value is the systolic blood pressure of the user and the blood pressure having the minimum value is the diastolic blood pressure of the user, from among the blood pressure calculated by the blood pressure calculator 1324. The blood pressure calculator 1324 transmits the systolic blood pressure and the diastolic blood pressure to the characteristic ratio calculator 1325, and calculates the characteristic ratio by using the systolic blood pressure, the diastolic blood pressure, and the sphygmus waves detected by the sphygmus wave detector 131. The characteristic ratio calculator 1325 calculates a ratio of the amplitude during the systolic blood pressure to the maximum amplitude, and a ratio of the amplitude during the diastolic blood pressure to the maximum amplitude, by using the sphygmus waves filtered by a HPF. In an example, the amplitudes during the systolic blood pressure and the diastolic blood pressure are amplitudes at a time when the pressure shown in the sphygmus waves filtered by the LPF is identical to the systolic blood pressure and the diastolic blood pressure. The ratio of the amplitude during the systolic blood pressure to the maximum amplitude is a systolic characteristic ratio, and the ratio of the amplitude during the diastolic blood pressure to the maximum amplitude is a diastolic characteristic ratio.

When the user is to newly estimate blood pressure by using the apparatus 1 according to an example, the blood pressure may be estimated by using the pre-calculated characteristic ratio, newly sensed values of sphygmus waves, and/or a newly determined MAP. The sphygmus wave detector 131 filters the newly sensed values of sphygmus waves via a HPF and a LPF to detect new sphygmus waves, and the pressure determiner 1321 determines a new MAP. Then, the characteristic ratio calculator 1325 estimates the blood pressure of the user by calculating the systolic blood pressure and the diastolic blood pressure using the newly detected sphygmus waves, the newly determined MAP, and the pre-calculated characteristic ratio.

The user may determine whether to estimate the blood pressure calculated by the blood pressure calculator 1324 by using one of Equations 1 through 12, or use the blood pressure calculated by using the characteristic ratio pre-calculated by the characteristic ratio calculator 1325 as the actual blood pressure of the user, according to a usage environment, for example. More particularly, the user inputs a method of estimating the blood pressure to the user interface 15. Thus, when the sensing unit 11 senses new values of sphygmus waves in the wrist while pressurizing the wrist with the variable pressure, the blood pressure calculator 1324 may calculate and estimate the systolic blood pressure and the diastolic blood pressure by using Equations 1 through 12, or, alternatively, using the characteristic ratio pre-calculated based on the newly sensed values of sphygmus waves.

The hydrostatic pressure change calculator 133 calculates a hydrostatic pressure change between two height positions using information input via the user interface 15 or, alternatively, using information stored in the storage unit 14. In an example, the input information includes a height difference between the height positions, a blood density and/or physical information of the user, for example. As described in greater detail above with reference Equation 7, the hydrostatic pressure change is calculated by multiplying the blood density p, the acceleration of gravity g and the height difference h between the first height position and the second height position.

The hydrostatic pressure change calculator 133 obtains the blood density ρ is stored in the storage unit 14 (or that is inputted through the user interface 15 by the user). In general, blood density of a person is typically about 1.06 grams per cubic centimeter (g/cm³), but this value may be adjusted according to the user's selection. The hydrostatic pressure change calculator 133 may use a blood density of 1.06 g/cm³ as a default setting. However, if the user wants to select a different blood density level, a blood density level input through the user interface 15 by the user may be inputted.

The hydrostatic pressure change calculator 133 obtains a height difference h between the two height positions from the user interface 15 or, alternatively, from the storage unit 14. In an alternative example, however, a method of obtaining the height difference is determined according to the user's selection or a setting of the apparatus 1, as will now be described in further detail. It will be noted that alternative examples of a method of obtaining the height difference are not limited to those described herein.

In an example, the height difference h may be obtained by the user directly inputting the height difference h, by using a device for obtaining the height difference h, or by using a body measurement of the user, for example. If the height difference h inputted by the user is used, the hydrostatic pressure change calculator 133 obtains the height difference h input through the user interface 15. Thus, after blood pressure is estimated at the two height positions having the height difference h, the user inputs the height difference h through the user interface 15, and the hydrostatic pressure change calculator 133 obtains the input height difference h.

According to yet another alternative example, the user may use a string having one end connected to the apparatus 1, while another end thereof is connected to a weight, to determine the height difference based on a length of the string.

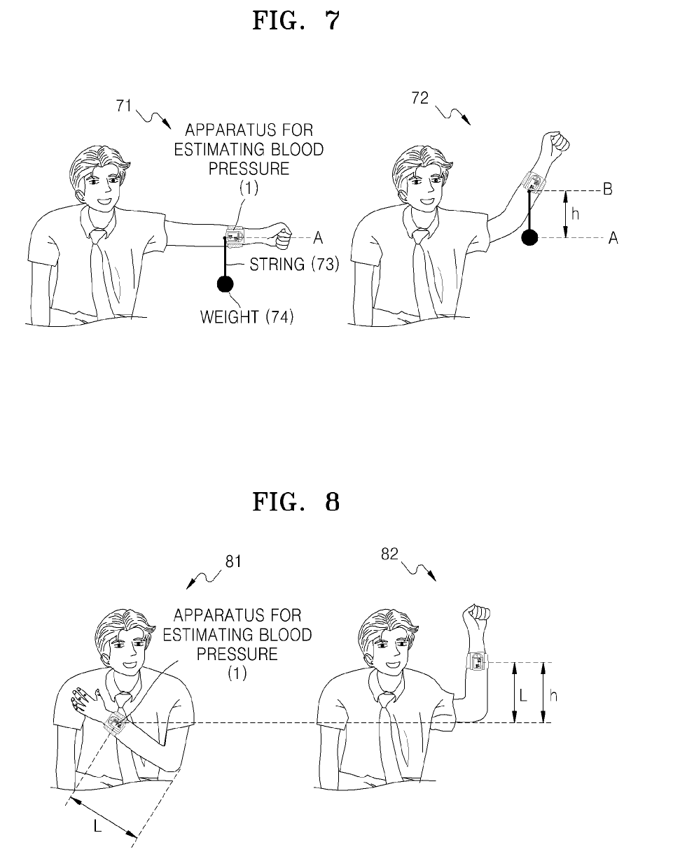

FIG. 7 is a diagram for describing using of a string 73 connected to a weight 74 for a user to determine a height difference h according to an alternative example. Referring to FIG. 7, a user's arm is located at each of an A height position and a B height position having a height difference h therebetween. To accurately recognize the A height position and the B height position, the user prepares the string 73 having the weight 74 at one end, so that a length from the center of the weight 74 to the apparatus 1 is identical to the height difference h, and thereafter connects the string 73 to the apparatus 1. Then, the user extends the arm straight at the A height position, as shown in drawing 71. Then, the user raises the arm until the center of the weight 74 is at the A height position, as shown in drawing 72. When the center of the weight 74 is at the A height position, the apparatus 1 is located at the B height position, wherein the A and B height positions differ by the height difference h. Accordingly, the user inputs the height difference h to the user interface 15, or adjusts the length of the string 73 to be the height difference h stored in the storage unit 14.

In an alternative example, the hydrostatic pressure change calculator 133 may obtain the height difference h by using the length of the user's arm, or by using the length of the user's arm and an accelerometer sensor. In this case, the user pre-inputs the arm length via the user interface 15 of the apparatus 1. However, when the user does not know the arm length, the arm length is statistically estimated by using physical information of the user, such as height, age and gender, for example. In an example, the arm length is from the elbow to the wrist. For example, when the user inputs their height and gender, for example, the apparatus 1 uses stored information about average arm lengths or average lengths from the wrist to the elbow of people having the same inputted height of the user.

FIG. 8 is a diagram for describing an alternative example of obtaining of a height difference h by using an arm length L. Referring to FIG. 8, the user puts on the apparatus 1, and the user's arm is positioned with the wrist on the chest and the end of the hand on the right shoulder, as shown in drawing 81. Then, the user positions the elbow at the same height as the shoulder, and bends the arm upward in a right angle, as shown in drawing 82. Thus, the height difference h is identical to the arm length L, and thus the height difference h is obtained by using the arm length L.

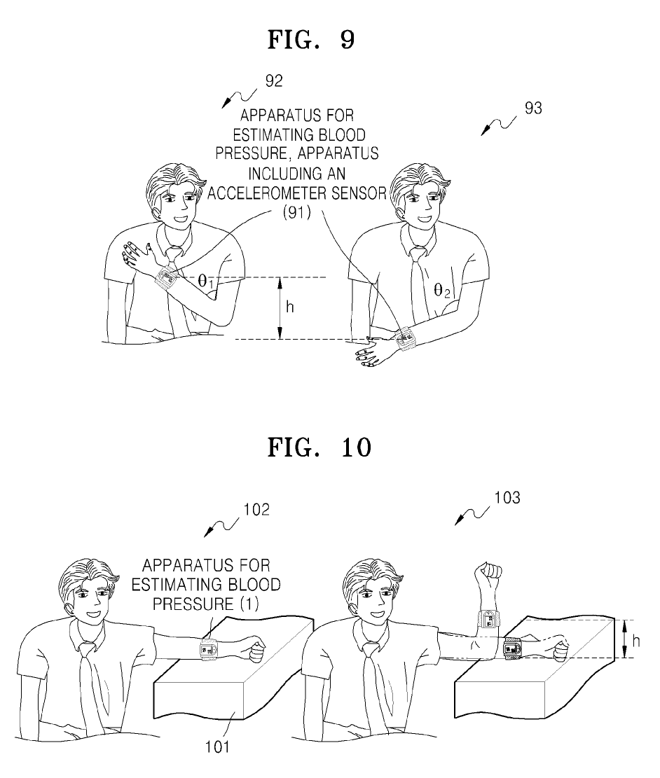

FIG. 9 is a diagram for describing obtaining of a height difference h by using an arm length and an accelerometer sensor according to another alternative example. Referring to FIG. 9, a user wears an apparatus 91 for estimating blood pressure, and the apparatus 91 includes an accelerometer sensor (not shown) on the wrist. The user places the wrist on which the apparatus 91 is worn near to the body, and measures an angle θ1 between the upper arm and the lower arm, as shown in drawing 92. Then, the user places the wrist near to the body in another position, and measures an angle θ2 between the upper arm and the lower arm, as shown in drawing 93. In an example, an angle difference based on a gravitational measurement, is determined by using the accelerometer sensor, and thus the angles θ1 and θ2 are determined. Accordingly, since the arm length L and the angles θ1 and θ2 are determined, the height difference h is obtained by using Equation 13 below.

h=L×(cos θ1−cos θ2) (Equation 13)

FIG. 10 is a diagram for describing obtaining of a height difference h by using an arm support 101 according to still another alternative example. Referring to FIG. 10, a user wears the apparatus 1 on the wrist, and sits down. The user's arm is positioned such that the arm is at a height of the shoulder, as shown in drawing 102. Then, the user raises the arm upwards to be at a right angle, as shown in drawing 103. According to the example of the method shown in FIG. 10, the blood pressure of the user is estimated at two height positions having the height difference h. Thus, the height difference h is equivalent to a distance from the wrist to the elbow of the user.

Referring again to FIG. 1, the user interface 15 according to an example receives information about blood density, a height difference and physical information, for example, from the user, or outputs information about a result of estimating blood pressure to the user. The result of estimating blood pressure is a result estimated based on a calculation result of the blood pressure calculator 1324 and/or a calculation result of the characteristic ratio calculator 1325. The user interface 15 obtains information from the user using any type of information input device or method, for example, a keyboard, a mouse, a touch screen and/or speech recognition, for example, while alternative examples are not limited thereto. Thus, the apparatus 1 according to an example obtains information, such as a height difference between the height positions where blood pressure is estimated blood density, physical information, and the like through the user interface 15, according to the user's selection or a setting of the apparatus 1. Also, the user may input a desired method of calculating blood pressure to the user interface 15, to determine how the estimator 132 will estimate the user's blood pressure. In other words, the user may determine which one of the blood pressure calculated by the blood pressure calculator 1324 or the blood pressure calculated by the characteristic ratio calculator 1325 will be estimated as the actual blood pressure. In addition, the user interface 15 includes a devices which displays visual information such as a display, a liquid crystal display (“LCD”) screen, a light-emitting-diode (“LED”) display or a division display device, for example, and/or devices providing auditory information such as speakers, for example.

In an example, the storage unit 14 stores any or all results performed, processed and/or obtained from the sensing unit 11, the pressurizer 12, the processor 13, the user interface 15, the actuator 16 and the controller 17. Also, the sensing unit 11, the pressurizer 12, the processor 13, the user interface 15, the actuator 16 and the controller 17 may read information stored in the storage unit 14. The processor 13 includes the sphygmus wave detector 131, the estimator 132 and the hydrostatic pressure change calculator 133, and the storage unit 14 stores any or all results performed, processed and/or obtained from elements in the processor 13.

The controller 17 controls an operation of the sensing unit 11, the processor 13, the storage unit 14, the user interface 15 and the actuator 16.

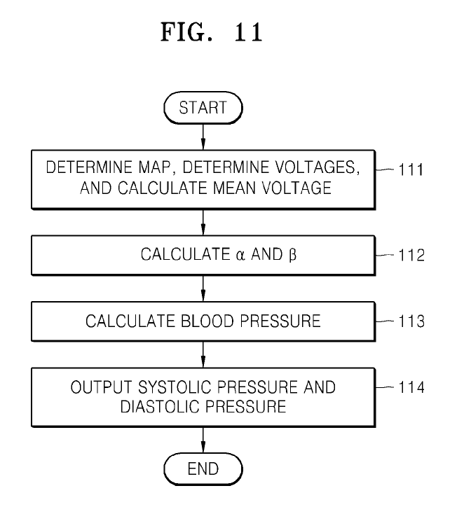

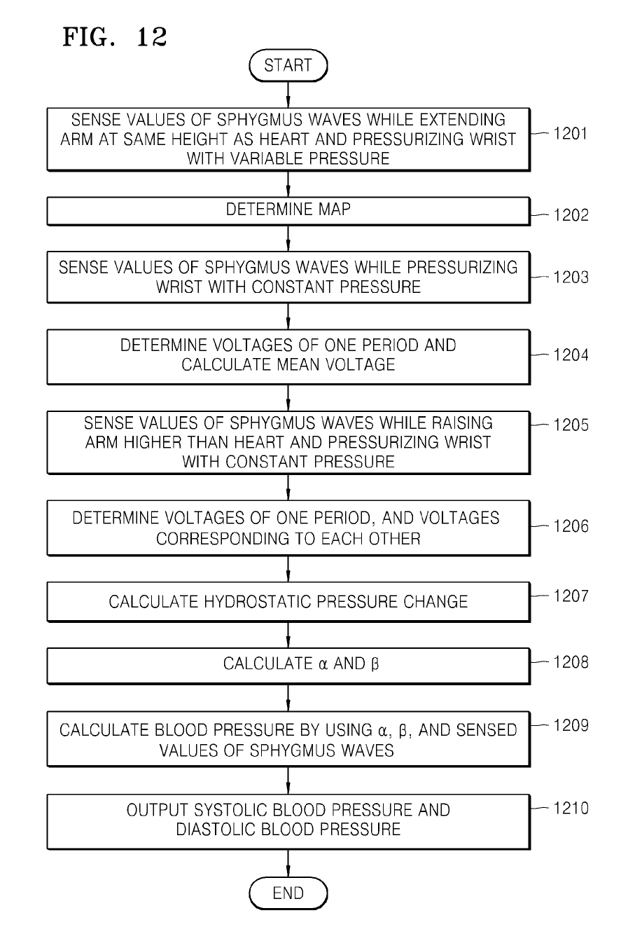

FIG. 11 is a flowchart illustrating an example of a method of estimating blood pressure. Referring to FIG. 11, the method according to an example includes operations, e.g., steps, performed sequentially with or by the apparatus 1 shown in FIG. 1.

In step 111, the pressure determiner 1321 determines the MAP in sphygmus waves, the voltage determiner 1322 determines voltages of one period in the sphygmus waves, and voltages corresponding to each other, and the voltage calculator 1323 calculates a mean voltage.

In step 112, the blood pressure calculator 1324 calculates α and β of Equation 4, above.

In step 113, the blood pressure calculator 1324 calculates blood pressure by using Equation 4 based on α, β, and voltages corresponding to values of the sphygmus waves.

In step 114, the user interface 15 outputs blood pressure having the maximum value as systolic blood pressure, and blood pressure having the minimum value as diastolic blood pressure.

FIG. 12 is a flowchart illustrating an order of estimating blood pressure of a user by using an example of a method of estimating blood pressure.

In step 1201, a user extends their arm to a first height position having substantially the same height as the user's heart. The pressurizer 12 pressurizes the wrist of the user with a first pressure, e.g., a variable pressure, and the sensing unit 11 senses values of a first sphygmus wave while the wrist is pressurized with the first pressure.

In step 1202, the pressure determiner 1321 determines the MAP.

In step 1203, the pressurizer 12 pressurizes the wrist with a second pressure, e.g., a constant pressure, and the sensing unit 11 senses values of a second sphygmus wave while the wrist is pressurized with the second pressure.

In step 1204, the voltage determiner 1322 determines voltages of one period of the first and/or second sphygmus waves, and the voltage calculator 1323 calculates a mean voltage of the voltages of one period.

In step 1205, the user raises their arm to a second height position that is higher than the first height position. The pressurizer 12 pressurizes the wrist with the second pressure, and the sensing unit 11 senses values of sphygmus waves while the wrist is pressurized with the second pressure.

In step 1206, the voltage determiner 1322 determines voltages of one period of the sphygmus waves, and voltages corresponding to each other from among the voltages of one period determined at the first and second height positions.

In step 1207, the hydrostatic pressure change calculator 133 obtains a height difference between the first and second height positions to calculate a hydrostatic pressure change of blood of the user.

In step 1208, the blood pressure calculator 1324 calculates α and β by using the MAP, the voltages corresponding to each other, the mean voltage and the hydrostatic pressure change.

In step 1209, the blood pressure calculator 1324 calculates blood pressure by using α, β and voltages corresponding to the values of the sphygmus waves. The estimator 132 estimates the calculated blood pressure as the actual blood pressure of the user.

In step 1210, the user interface 15 outputs the blood pressure having the maximum value as a systolic blood pressure, and the blood pressure having the minimum value as a diastolic blood pressure.

As described herein, according to one or more examples, blood pressure is accurately estimated, since a statistical characteristic ratio, which typically includes an error according to the race, gender or age, for example, of a user, is not required. Additionally, the blood pressure may be continuously estimated in an example.

Thus, in the examples described herein, an applied pressure on the wrist for measuring blood pressure is slightly higher than a MAP and much lower than an artery occlusion pressure. In the conventional volume oscillometric blood pressure measurement method, however, an applied pressure on the wrist is higher than an artery occlusion pressure. Therefore, a blood pressure measurement using low pressurization is available, which is desirable for user convenience.

The examples described herein can be written as computer programs and can be implemented in general-use digital computers that execute the programs using a computer readable recording medium, for example. Data used in the above-described examples can be recorded on a medium in various forms. Examples of computer readable recording medium include magnetic storage media, e.g., read only memory (“ROM”) floppy disks and hard disks, as well as optical recording media such as Compact Disk-Read Only Memory (“CD-ROM”) or (digital versatile disc “DVD”), for example.

It will be understood that the examples described herein should be considered in a descriptive sense only, and not for purposes of limitation. Descriptions of features or aspects within each example should typically be considered as available for other similar features or aspects in alternative examples.

While the present invention has been particularly shown and described with reference to examples thereof, it will be understood by those of ordinary skill in the art that various changes in form and details may be made therein without departing from the spirit or scope of the present invention as defined by the following claims.

What is claimed is:

1. A method of estimating blood pressure, the method comprising:
sensing a value of a first sphygmus wave in a region of a user's body while pressurizing the region with a first pressure;
sensing a value of a second sphygmus wave in the region while pressurizing the region with a second pressure; and
estimating blood pressure of the region based on sensed values of the first sphygmus wave and the second sphygmus wave, wherein
the first pressure and the second pressure are each one of a variable pressure and a constant pressure, and
a height of the region, relative to the user's body, is different for the sensing the value of the first sphygmus wave than for the sensing the value of the second sphygmus wave.

2. The method of claim 1, wherein the estimating the blood pressure of the region comprises:
determining a value of the first pressure at a point of time when a value of the first sphygmus wave sensed in the region pressurized with the variable pressure, has an estimated maximum amplitude or a maximum amplitude interpolated by using peak values of the first sphygmus wave; and
calculating the blood pressure in the region by using the value of the first pressure and the sensed values of the first sphygmus wave and the second sphygmus wave while pressurizing the region with the second pressure, which is the constant pressure.

3. The method of claim 2, wherein the estimating the blood pressure of the region further comprises:
determining voltages of a first period of the first sphygmus wave from a plurality of voltages corresponding to the sensed values of the first sphygmus wave and the second sphygmus wave while pressurizing the region with the second pressure at a first height position and a second height position; and
calculating a mean of the voltages of the first period at one of the first height position and the second height position,
wherein
in the calculating the blood pressure, the blood pressure of the region is calculated by using the value of the first pressure, the voltages of the first period and the mean of the voltages of the first period,
the first height position corresponds to a height at which one of the value of the first sphygmus wave and the value of the second sphygmus wave is sensed, and
the second height position corresponds to a height at which another of the one of the value of the first sphygmus wave and the value of the second sphygmus wave is sensed.

4. The method of claim 3, wherein
the determining the voltages comprises determining corresponding voltages from a plurality of the determined voltages of the first period at each of the first height position and the second height position, and
the calculating the blood pressure comprises:
calculating a first value by using a hydrostatic pressure change of blood according to a height difference between the first height position and the second height position and a difference between the corresponding voltages;
calculating a second value by using the first value, the value of the first pressure and the mean of the voltages of the first period; and
calculating the blood pressure in the region by using the first value and the second value, and voltages corresponding to the sensed values of the first sphygmus wave and the second sphygmus wave.

5. The method of claim 4, wherein the hydrostatic pressure change of the blood is calculated by using at least one of a height difference, physical information and a blood density inputted by the user.

6. The method of claim 3, wherein
in the determining the value of the first pressure, the value of the first pressure is determined at a point of time when the value of the first sphygmus wave sensed in the region being pressurized with the variable pressure is expected to have one of the maximum amplitude and the value interpolated by using the peak values of the first sphygmus wave at each of the first height position and the second height position,
in the calculating the mean, the mean of the determined voltages of one period is calculated at each of the first height position and the second height position, and
the calculating the blood pressure comprises:
calculating a first value and a second value by using the determined pressure and the calculated mean in each of the first height position and the second height position; and
calculating the blood pressure in the region by using the calculated first value and the calculated second value, and voltages corresponding to the sensed values of the first sphygmus wave and the second sphygmus wave.

7. The method of claim 1, wherein the variable pressure is one of a continuously increasing pressure, a continuously decreasing pressure and two or more discrete constant pressures varied in a stepwise form.

8. The method of claim 1, further comprising:
estimating a plurality of blood pressures of the region;
outputting a blood pressure of the plurality of blood pressures having a maximum value as a systolic blood pressure; and
outputting a blood pressure from the plurality of blood pressures having a minimum value as a diastolic blood pressure.

9. The method of claim 2, wherein the estimating the blood pressure further comprises calculating a characteristic ratio of the user's blood pressure by using the sensed values of the first sphygmus wave, the second sphygmus wave and the calculated blood pressure while pressurizing the region with the variable pressure, and
the blood pressure in the region is estimated based on the characteristic ratio.

10. The method of claim 5, wherein
when the physical information comprises a length of the user's arm, the height difference between the first height position and the second height position is obtained by using the length of the user's arm and a difference of angles formed when the user's arm at the first height position and the second height position, and
when the physical information does not include the user's arm length, the arm length is estimated by using the physical information which does not include the user's arm length.

11. A computer program product comprising:
a computer readable computer program code which stores and implements a method of estimating blood pressure; and
instructions for causing a computer to implement the method, the method comprising:
sensing a value of a first sphygmus wave in a region of a user's body while pressurizing the region with a first pressure;
sensing a value of a second sphygmus wave in the region while pressurizing the region with a second pressure; and
estimating blood pressure of the region based on sensed values of the first sphygmus wave and the second sphygmus wave, wherein
the first pressure and the second pressure are each one of a variable pressure and a constant pressure, and
a height of the region, relative to the user's body, is different for the sensing the value of the first sphygmus wave than for the sensing the value of the second sphygmus wave.

12. An apparatus for estimating blood pressure, the apparatus comprising:
a sensing unit which senses a value of a first sphygmus wave in a region of a user's body while pressurizing the region with a first pressure, and which senses a value of a second sphygmus wave in the region while pressurizing the region with a second pressure;
an estimator which estimates blood pressure of the region based on sensed values of the first sphygmus wave and the second sphygmus wave; and
a user interface which outputs the blood pressure of the region, wherein
the first pressure and the second pressure are each one of a variable pressure and a constant pressure, and
a height of the region, relative to the user's body, is different for the sensing the value of the first sphygmus wave than for the sensing the value of the second sphygmus wave.

13. The apparatus of claim 12, wherein the variable pressure is one of a continuously increasing pressure, a continuously decreasing pressure and two or more discrete constant pressures varied in a stepwise form.

14. The apparatus of claim 12, wherein the estimator comprises:
a pressure determiner which determines a value of the first pressure at a point of time when a value of the first sphygmus wave sensed in the region pressurized with the variable pressure has an estimated maximum amplitude or a maximum amplitude interpolated by using peak values of the first sphygmus wave; and
a blood pressure calculator which calculates the blood pressure in the region by using the value of the first pressure and the sensed values of the first sphygmus wave and the second sphygmus wave while pressurizing the region with the constant pressure.

15. The apparatus of claim 14, wherein the estimator further comprises:
a voltage determiner which determines voltages of a first period of the sphygmus wave from a plurality of voltages corresponding to the sensed values of the first sphygmus wave and the second sphygmus wave while pressurizing the region with the constant pressure, at a first height position and a second height position; and
a voltage calculator which calculates a mean of the determined voltages in one of the first height position and the second height position,
wherein the blood pressure calculator calculates the blood pressure of the region by using the determined pressure, the determined voltages of one period and the calculated mean of the determined voltages.

16. The apparatus of claim 15, wherein the voltage determiner determines corresponding voltages from among a plurality of the determined voltages of the first period at each of the first height position and the second height position, and
the blood pressure calculator calculates a first value by using a hydrostatic pressure change of blood according to a height difference between the first height position and the second height position and a difference between the corresponding voltages, calculates a second value by using the first value, the determined pressure and the calculated mean, and calculates the blood pressure in the region by using the first value and the second value, and voltages corresponding to the sensed values of the first sphygmus wave and the second sphygmus wave.

17. The apparatus of claim 15, wherein the pressure determiner determines pressure at a point of time when the value of the first sphygmus wave sensed in the region being pressurized with the variable pressure has the maximum amplitude at each of the first height position and the second height position,
the voltage calculator calculates the mean of the determined voltages of one period at each of the first height position and the second height position, and
the blood pressure calculator calculates a first value and a second value by using the determined pressure and the calculated mean at each of the first height position and the second height position, and calculates the blood pressure in the region by using the first value, the second value, and voltages corresponding to the sensed values of the first sphygmus wave and the second sphygmus wave.

18. The apparatus of claim 12, wherein
the estimator estimates a plurality of estimated blood pressures in the region, and
the plurality of estimated blood pressures comprises:
a systolic blood pressure which has a maximum value of the plurality of estimated blood pressures; and
a diastolic blood pressure which has a minimum value of the plurality of estimated blood pressures.

19. The apparatus of claim 14, wherein the estimator further comprises a characteristic ratio calculator, which calculates a characteristic ratio of the user's blood pressure by using the sensed values of the first sphygmus wave, the second sphygmus wave and the calculated blood pressure while pressurizing the region with the variable pressure, and
the blood pressure in the region is estimated based on the characteristic ratio while pressurizing the region with the variable pressure.

20. The apparatus of claim 16, further comprising a member, wherein the height difference between the first height position and the second height position is determined based on movement along a length of the member.
